(19) United States
(12) Patent Application Publication
(10) Pub. No.: US 2007/0249537 A1
(43) Pub. Date: Oct. 25, 2007

(54) ABSORPTION ENHANCING AGENTS

(75) Inventors: Rong-Kun CHANG, Rockville, MD (US); Benjamin Kibalo, Borough, NJ (US); Richard A. Couch, Bryn Mawr, PA (US); Mark J. Ginski, Perry Hall, MD (US); Ali Keshavarz-Shokri, San Diego, CA (US); Caren C. Bancroft, Germantown, MD (US)

Correspondence Address: FOLEY AND LARDNER LLP;SUITE 500, 3000 K STREET NW, WASHINGTON, DC, 20007, US

(73) Assignee: Supernus Pharmaceuticals, Inc.

(21) Appl. No.: 11/741,355

(22) Filed: Apr. 27, 2007

Related U.S. Application Data
(63) Continuation-in-part of application No. 10/762,446, filed on Jan. 22, 2004, now abandoned.
(60) Provisional application No. 60/441,950, filed on Jan. 23, 2003.

(51) Int. Cl. A61K 47/12; A61K 31/549; A61K 31/7052; A61P 43/00; A61K 38/00; A61K 38/16

(52) U.S. Cl. 514/12; 514/2; 514/223.2; 514/44; 514/784

(57) ABSTRACT

Disclosed are new compounds that increase the absorption of pharmaceutical agents across mucous membranes. These absorption enhancers allow higher bioavailability of administered drugs. The enhancers advantageously have low or no cytotoxicity.

### CROSS-REFERENCE TO RELATED PATENT APPLICATIONS

This application is a continuation-in-part of the U.S. patent Application Ser. no. 10/762446, filed on Jan. 22, 2004, which claims the benefit of U.S. Provisional Application No. 60/441,950, filed Jan. 23, 2003, incorporated herein by reference in its entirety.

### FIELD OF THE INVENTION

The present invention is directed to pharmaceutical compositions that contain, or are administered together with, certain mucosal membrane absorption enhancing compounds. The compositions beneficially increase the bioavailability of the active pharmaceutical agent or agents in the composition.

### BACKGROUND OF THE INVENTION

Many drugs are administered in a manner that requires the therapeutic agent to cross a mucosal membrane cellular layer. These drugs face factors limiting the bioavailability, and thus the therapeutic performance, of the active agent. For instance, mucosal layers of epithelium are encountered when administering drugs orally, sublingually, buccally, rectally, intranasally, vaginally, and ocularly.

Most systemic drugs are administered enterally, intranasally or by inhalation for patient comfort reasons. “Enterally” for the purposes of this disclosure means any way of administration whereby the drug is absorbed through the gastrointestinal tract, including the oral mucosa. In order for enterally administered drugs to have a systemic effect, they must somehow pass from the lumen of the GI tract to the underlying circulation. The epithelial cells lining the GI tract present a barrier to the efficient absorption of enterally administered drugs. Similarly, the epithelial cells forming the lining of the respiratory system are an obstacle to the efficient absorption of intranasal or inhaled administration. Drug compositions that have the ability to enhance the transport of drugs across the mucosal membranes of various body cavities would be an improvement in the pharmaceutical arts.

It has been found that when poorly absorbed drugs are administered orally or rectally, for instance, the bioavailability of the drugs could be increased by administering them together with absorption enhancer(s). However, most of these enhancers, e.g., sodium salicylate, 5-methoxysalicylate, sodium cholate, S-nitroso-N-acetyl-DL-penicillamine, sodium benzoate, sodium gentisate, sodium lauryl sulfate, etc., can damage and irritate the intestinal mucosal membrane. Therefore, there remains a need in the field for effective, but safe, absorption enhancers.

### SUMMARY OF THE INVENTION

In one aspect, the present invention is directed to a composition comprising at least one pharmaceutically active agent and one or more absorption enhancer selected from thioctic acid, sebacic acid, shikimic acid, N-alkylated amino acids and salts thereof, and methods of preparing the same. In the preferred embodiment of the invention, N-alkylated amino acid is selected from N,N-dimethylglycine and trimethylglycine.

A further aspect of the present invention is a method for enhancing the absorption of a pharmaceutically active agent or agents through mucous membranes of body cavities, comprising administering to the body cavity a combination comprising at least one active agent and one or more absorption enhancer selected from thioctic acid, sebacic acid, shikimic acid, N-alkylated amino acids and salts thereof.

### DETAILED DESCRIPTION OF THE PREFERRED EMBODIMENTS

With the present invention it was found that thioctic acid, sebacic acid, shikimic acid and N-alkylated amino acids consistently improved the permeability of mannitol, sampatrilat and hydrochlorothiazide across a Caco-2 cell line that forms a confluent epithelial layer. In addition, these new excipients have low cytotoxicity.

The Caco-2 cell line is a well-recognized in vitro screening model, which both structurally and functionally represents the small intestinal epithelium. Caco-2 cells are derived from human colon carcinoma cells and differentiate in culture to form intestinal epithelia similar to that found in the small intestine. More specifically, Caco-2 cells form a brush border with normal enzymes, form tight junctions between cells, and acquire the barrier properties of an enterocyte sheet. This cell line was utilized to evaluate the absorption enhancers and drug formulations of the present invention in a manner known and which is generally disclosed, for example, in Drug Absorption Enhancement, A. (Bert) G. de Boer, ed., ISBN 3-7186-5492-X (1994), which is incorporated herein by reference, particularly Chapter 3 thereof.

In addition, a lactate dehydrogenase (LDH) assay was conducted after the permeation studies to evaluate the cytotoxicity of the absorption enhancers as well as to discover any violation of the integrity of the Caco-2 cells. LDH is a cytosolic enzyme that is not normally secreted outside the cell. However, it leaks into the culture medium upon damage to the cell membranes. In vitro release of LDH from cells provides an accurate measure of cell membrane integrity and cell viability. Although used in immunological studies and in studies that test the biocompatibility of implantable biomaterials, the present inventors have found that it is a reliable and accurate test of the cellular toxicity of pharmaceutical excipients such as the enhancers of this invention. Wu, S.-J., et al, Pharmaceutical Res., 16(8): 1266-1272 (1999); Allen, M. J. et al., Promega Notes Magazine, Number 45, p. 7 (1994); or Ehrlich, M. et al, Current Protocols in Toxicology, John Wiley & Sons, New York (2000). LDH leakage into the apical compartment of the Caco-2 cell system was used to measure the effect, if any, of a given absorption enhancer on the viability of the Caco-2 cells.

Compositions according to the present invention are comprised of one or more pharmaceutically active agents, and one or more of the enhancer excipients selected from thioctic acid, sebacic acid, shikimic acid, N-alkylated amino acids and salts thereof. Preferably, N-alkylated amino acid is a N-methylated amino acid. The amino acid is selected from a group consisting of isoleucine, leucine, lysine, methionine, phenylalanine, threonine, tryptophan, valine, arginine, histadine, alanine, asparagine, cysteine, glutamate, glutamine, glycine, proline, serine, and tyrosine. In a most preferred embodiment, N-alkylated amino acid is N,N-dimethylglycine or trimethylglycine. Glycine is an amino acid found in the protein of all life forms. Glycine and methylated glycines are very safe compounds, abundantly represented in a normal diet, and high doses of these compounds are well tolerated. In addition, these materials possess some beneficially physiological and pharmacological effects toward the human bodies.

The pharmaceutical active agent of the current invention is any drug, either now known or later discovered, that could benefit from enhanced absorption when advantageously formulated with the enhancers of the present invention. Typically, it would be a drug that exhibits poor bioavailability due to poor permeation of a mucosal epithelial cell layer, such as in the gastrointestinal tract, which would include inter alia such active agents as peptides, proteins and nucleic acids. The present compositions are not limited to a particular drug or combination of drugs, and it is contemplated that the enhancers have widespread applicability. For purposes of demonstration herein there are disclosed formulations of the enhancers with two drugs known for their poor bioavailability, sampatrilat and hydrochlorothiazide, but the invention should not be considered as limited to these exemplary embodiments. In fact, the inventors have found that, along with mannitol, these two drugs are useful for screening additional absorption enhancer excipients. Though the pharmaceutically active agent in the composition may be utilized in the typical therapeutic amount, it is anticipated that a smaller dose will be sufficient because of the enhanced bioavailability.

The compositions of the present invention can contain just one of the enhancers, or a combination of two or more. In general, the enhancers are present in an amount effective to act as an absorption enhancer of the administered drug or drugs, and this amount can be estimated empirically. An amount effective can be one that increases the bioavailability of the drug to any appreciable extent. The enhancers can be present in a concentration in the final dosage form of from about 0.01% to about 99% by weight, alone or in combination. Preferably, the enhancers are present in the final composition at about 0.01% to about 50% by weight, and more preferably about 0. 1% to about 30% by weight. The optimal amount in a given formulation can, of course, be estimated or determined by experimentation such as that described in the examples.

The compositions are in a form suitable for oral, nasal, buccal, sublingual, topical, rectal, or vaginal administration, and may be in the form of liquids, solids, lotions, gels, aerosols, or any other pharmaceutical vehicle. For oral administration, the compositions may be in the form of liquids, suspensions, emulsions, powders, pills, tablets, capsules, gel caps, troches, cachets, pellets, and the like. With pharmaceutically suitable liquids the compositions can take the form of a solution, suspension (or dispersions), aerosol or emulsion, which can be sprayed or inhaled.

The formulations may be prepared by any methods well known in the art of pharmacy, for example, using methods such as those described in Gennaro et al., Remington's Pharmaceutical Sciences (18th ed., Mack Publishing Company, 1990, see especially Part 8: Pharmaceutical Preparations and their Manufacture). Such methods comprise the step of bringing into association the drug(s), pharmaceutical carrier and enhancer(s). Prior to admixing with the pharmaceutical agent and accessory ingredients (if desired), the enhancer may be solubilized in an appropriate solvent system, such that the final concentration of enhancer(s) in the compositions of the present invention is between about 0.01% to about 99% by weight, preferably about 0.1% and about 50% by weight, and more preferably between about 0.1% and about 30% by weight. Pharmaceutical carriers are suitable vehicles in which the drug or drugs (or “pharmaceutically active agent”) are incorporated in by dissolving, dispersing, or suspending, and include such vehicles as, for example, solvents, lipids, proteins, carbohydrates, polymers, etc., and substances that are added to increase solubility or dispersion of the active agent, such as solubilizers, emulsifiers, and surfactants, for instance. Other accessory ingredients include those conventional in the art, such as fillers, binders, diluents, disintegrants, glidants, lubricants, colorants, flavoring agents and wetting agents.

Preferred embodiments of the invention are those compositions that are administered orally, and which increase the absorption of the active ingredient(s) in the gastrointestinal tract. For oral administration, the compositions may be in the form of liquids, suspensions, emulsions, powders, pills, tablets, capsules, troches, cachets, pellets, effervescent powders or granules, gel caps, and the like. These dosage forms are prepared in manners known in the art, such as disclosed in Gennaro et al., Remington's Pharmaceutical Sciences, supra.

A further aspect of the present invention is a method for enhancing the absorption across a mucosal membrane of a pharmaceutically active agent, which comprises administering a composition comprising the active agent (or agents) and one or more of thioctic acid, sebacic acid, shikimic acid, N-alkylated amino acids, or their salts.

Preferably, N-alkylated amino acid is an N-methylated amino acid. In the preferred embodiment, the N-alkylated amino acid is a N-methylated amino acid selected from a group consisting of isoleucine, leucine, lysine, methionine, phenylalanine, threonine, tryptophan, valine, arginine, histadine, alanine, asparagine, cysteine, glutamate, glutamine, glycine, proline, serine, and tyrosine. In the most preferred embodiment, the enhancer is selected from a group consisting of N,N-dimethylglycine, trimethylglycine and salts and combinations thereof.

The use of the permeation enhancers of the invention to promote mucosal membrane absorption affords several advantages over the absorption promoting compounds described in the prior art. The permeation enhancers of the current invention are more potent than the currently available absorption promoting agents. As an example, at 1% w/v concentration, thioctic acid can effectively enhance hydrochlorothiazide permeability across a Caco-2 monolayer 13-fold more than the patented permeation enhancer, 18.beta.-glycyrrhetinic acid. This difference in potency allows opportunities for reducing the required size of the dosage form and potentially minimizing side effects. Additionally, the results from the lactose dehydrogenase assay reveal that the enhancers (i.e., methylated glycines, thioctic acid, sebacic acid, shikimic acid) are not cytotoxic relative to cells treated with Hank's balanced salt solution alone.

### EXAMPLES

### Example 1

Sampatrilat is a hydrophilic compound containing one weakly acidic phenolic group, two more strongly acidic carboxylic acid groups, and one strong basic primary amine group with an aqueous solubility of 1.8 mg/mL. The compound has relatively low oral bioavailability, primarily due to its poor intestinal permeability. Earlier studies demonstrated about 2-5% oral bioavailability in vivo when administered by a tablet dosage form. Thus, sampatrilat is a good low permeability model drug.

In this example and Example 2, Caco-2 cells were grown to confluence on permeable supports mounted in a chamber that has an apical (AP) side and a basolateral (BL) side. Sampatrilat and enhancer were added to the apical chamber to give a concentration of 1.8 mg/mL and 1% w/v, respectively. Permeability coefficients are determined as previously reported by Yazdania et.el (Yazdanian M, Glynn, S I, Wright J L, et al. 1998. Correlating partitioning and Caco-2 permeability of structurally diverse small molecular weight compounds. Pharm Res 15:1490-1494). Briefly, drug solutions were prepared in HBSS at a known final concentration. For AP to BL experiments, the solution was placed on the apical side of the cells and samples were taken from basolateral side. In contrast, for BL to AP experiments, the solution was placed on the basolateral side of the cells and samples were taken from apical side. The samples are analyzed by an HPLC. Transport rates (J) are determined by plotting cumulative amounts of drug permeated as a function of time. Apparent permeability coefficients P.sub.caco-2, are determined according to the equation P.sub.caco-2=J/ACi where Ci is the initial concentration of the solution in donor chamber and A is the surface area of the filter.

Table 1 shows the calculated permeability coefficients from the Caco-2 transport study. N,N-dimethylglycine, thioctic acid, sebacic acid, and shikimic acid significantly increase the sampatrilat permeation across the Caco-2 cell line. As an example, N,N-dimethylglycine increases sampatrilat permeability 124-fold over the drug alone. The original cell line integrity and the effect of excipients on the integrity of cell line were also tested by measuring the flux of ¹⁴C-mannitol. Except for thioctic acid, it is clear from the data that markedly enhanced transport of sampatrilat by N,N-dimethylglycine, sebacic acid, and shikimic acid coincided with the increased transport of mannitol. Although not intending to be bound to any particular theory, the parallel-enhanced transport of mannitol may indicate that N,N-dimethylglycine, sebacic acid, and shikimic acid increases the paracellular permeation of sampatrilat by opening the tight junctions within the epithelial barrier.

TABLE 1
Permeability coefficients of Sampatrilat transport across Caco-2 cell line

| Permeability Coefficient, 10E−6 cm/s | | | | Enhancement Ratio | | |
|---|---|---|---|---|---|---|
| Compound | | Sampatrilat | Mannitol | Sampatrilat | Mannitol | |
| Control | No Drug | N/A | 4.6 | — | — | Toxicity |
| PD0058- | Drug | 0.16 | 0.49 | 1 | 1 | No |
| 152A | alone¹ | | | | | |
| PD0058- | Sebacic | 2.10 | 7.50 | 13.1 | 15.3 | No |
| 152B | acid² | | | | | |
| PD0058- | Amino | 0.48 | 1.07 | 3.0 | 2.2 | Yes |
| 152C | caproic | | | | | |
| | acid² | | | | | |
| PD0058- | N,n- | 19.90 | 12.30 | 124.4 | 25.1 | No |
| 152D | dimenthylglycine² | | | | | |
| PD0058- | Thioctic | 16.60 | 0.60 | 103.8 | 1.2 | No |
| 152E | acid² | | | | | |
| PD0058- | Citrulline² | 0.34 | 0.22 | 2.1 | 0.45 | Yes |
| 152F | | | | | | |
| PD0058- | Kojic acid² | 0.49 | 1.63 | 3.1 | 3.3 | Yes |
| 152G | | | | | | |
| PD0058- | Shikimic | 3.65 | 15.80 | 22.8 | 32.4 | No |
| 152H | acid² | | | | | |

¹Sampatrilat concentration at 1.8 mg/mL was used for all the Caco-2 transport studies.
²The concentration at 1% w/v was used for all the excipients in this Caco-2 study.

### Example 2

Hydrochlorothiazide is another known low permeability compound. Again, N,N-dimethylglycine, thioctic acid, sebacic acid, and shikimic acid were demonstrated as permeability enhancers in the Caco-2 transport studies using hydrochlorothiazide as a model drug (Table 2). Additionally, the results from the lactose dehydrogenase assay reveal that the excipients (i.e., N,N-dimethylglycine, thioctic acid, sebacic acid, shikimic acid) are not cytotoxic relative to cells treated with Hank's balanced salt solution alone.

Several patented absorption-promoting agents (e.g., cyclopentadecanolide, U.S. Pat. Nos. 5,731,303 and 5,023,252; glycyrrhetinic acid, U.S. Pat. No. 6,214,378; piperine, U.S. Pat. No. 5,616,593; and Vitamin E TPGS, U.S. Pat. Nos. 5,891,845 and 5,234,695) were examined for their permeability enhancing effect and are also shown in Table 2. As can be seen, these agents show low or no potency in permeability enhancement, compared to the agents of the present invention.

TABLE 2
Permeability coefficients of hydrochlorothiazide transport across Caco-2 cell line

| | | | Permeability | | |
|---|---|---|---|---|---|
| | | Study | coefficient, | Enhancement | |
| Lot number | Sample description | number | (×10⁻⁶, cm/s) | Ratio | Toxicity |
| PD0058-161A | N,N-dimethylglycine | 1 | 24.2 | 9.6 | No |
| PD0058-161B | Thioctic acid | 1 | 25.2 | 10.0 | No |
| PD0058-161C | Cyclopentadecanolide | 1 | 2.03 | 0.8 | No |
| PD0058-161D | Drug alone | 1 | 2.53 | 1 | No |
| PD0058-161E | Glycyrrehetinic acid | 1 | 1.93 | 1.2 | No |
| PD0058-166C | Thioctic acid | 2 | 37.5 | 22.5 | No |
| PD0058-166E | Piperine | 2 | 1.42 | 0.9 | No |
| PD0058-166F | Drug alone | 2 | 1.67 | 1 | No |
| PD0058-166G | N,N-dimethylglycine | 2 | 34.7 | 20.8 | No |
| PD0058-167D | Sebacic acid | 3 | 7.30 | 12.2 | No |
| PD0058-167E | Shikimic acid | 3 | 13.5 | 22.6 | No |
| PD0058-167F | Vitamin E TGPS | 3 | 0.58 | 0.97 | No |
| PD0058-167H | Drug alone | 3 | 0.60 | 1 | No |
| PD0058-168B | Drug alone | 4 | 0.47 | 1 | No |
| PD0058-168F | N,N-dimethylglycine | 4 | 34.4 | 73.3 | No |
| PD0058-169A | Drug alone | 5 | 0.44 | 1 | No |
| PD0058-169E | Piperine¹ | 5 | 0.43 | 0.99 | Yes |
| PD0058-169G | Shikimic acid² | 5 | 36.4 | 82.7 | No |
| PD0058-169H | Cyclopentadecanolide¹ | 5 | 0.69 | 1.56 | No |

Note:
Hydrochlorothiazide concentration at 0.2 mg/mL was used for all the experiments.
Excipient concentration at 1% was used for the study #1 to #4; for the study #5, higher excipient concentration was tested.
¹5% w/v concentration
²3% w/v concentration
Enhancement ratio is the ratio of permeability coefficient of the test sample and permeability coefficient of mannitol control.

### Example 3

The effectiveness of N-methylated glycines as permeability enhancers was evaluated using mannitol as a control. The results are represented in Table 3;

TABLE 3
Permeability coefficients and enhancement ratios for the test samples
in comparison to the mannitol control

| | | Permeability | Enhance- | |
|---|---|---|---|---|
| | | coefficient | ment | |
| Lot number | Description | (×10⁻⁶ cm/sec) | ratio^{d} | Toxicity |
| API control | 6.8 uM Mannitol | 0.93^{a} | 1.0 | No |
| PD0200-55A | 1% glycine | 0.93 | 1.0 | No |
| PD0299-55B | 0.5% glycine | 0.85 | 0.9 | No |
| PD0200-55C | 1% dimethyl | 14.70 | 15.9 | No |
| | glycine | | | |
| PD0200-55D | 0.5% dimethyl | 9.22 | 9.9 | No |
| | glycine | | | |
| PD0200-55E | 1% sarcosine^{b} | 0.89 | 1.0 | No |
| PD0200-55F | 0.5% sarcosine | 1.71 | 1.8 | No |
| PD0200-55G | 1% betaine^{c} | 11.20 | 12.1 | No |
| API control | 6.8 uM Mannitol | 1.24 | 1.0 | No |
| PD0200-59A2 | 1% betaine and | 48.50 | 39.1 | No |
| | 1% Tween 80 | | | |
| PD0200-59A7 | 1% dimethyl | 46.30 | 37.3 | No |
| | glycine and 1% | | | |
| | Tween 80 | | | |

^{a}Mean (standard deviation);
^{b}Chemical name for sarcosine is methyl glycine;
^{c}Chemical name for betaine is trimethyl glycine;
Enhancement ratio is the ratio of permeability coefficient of the test sample and permeability coefficient of mannitol control.

What is claimed is:

1. A composition comprising at least one pharmaceutically active agent, and at least one enhancer, wherein said enhancer is an acid selected from a group consisting of N-alkylated amino acid, thioctic acid, sebacic acid, shikimic acid, and salts thereof, wherein the enhancer is present in the composition in an amount effective to increase the biological absorption of the active agent.

2. A composition of claim 1, wherein said N-alkylated amino acid is an N-methylated amino acid.

3. A composition of claim 2, wherein the amino acid is selected from a group consisting of isoleucine, leucine, lysine, methionine, phenylalanine, threonine, tryptophan, valine, arginine, histadine, alanine, asparagine, aspartate, cysteine, glutamate, glutamine, glycine, proline, serine, and tyrosine.

4. A composition of claim 3, wherein the amino acid is glycine.

5. A composition of claim 4, wherein said enhancer is selected from a group consisting of N,N-dimethylglycine, trimethylglycine and salts and combinations thereof.

6. A composition of claim 5, wherein said enhancer is N,N-dimethylglycine.

7. A composition of claim 1, wherein said enhancer is selected from a group consisting of thioctic acid, sebacic acid, shikimic acid, and salts thereof.

8. The composition of claim 3, wherein the concentration of the enhancer is from about 0.01% to about 99% by weight.

9. The composition of claim 4, wherein the concentration of enhancer is from about 0.01% to about 50% by weight.

10. The composition of claim 4, wherein the concentration of enhancer is from about 0.1% to about 30% by weight.

11. The composition of claim 1, wherein the active agent is a protein, peptide, or nucleic acid.

12. The composition of claim 1, wherein the active agent is selected from sampatrilat and hydrochlorothiazide.

13. The composition of claim 1, which is an oral pharmaceutical in the form of a liquid, suspension, emulsion, powder, pill, tablet, capsule, gel caps, troche, cachet or pellet.

14. The composition of claim 1, which is in the form of a solution, suspension, aerosol, or emulsion, which can be sprayed or inhaled.

15. A method for enhancing the absorption of a pharmaceutically active agent across a mucosal membrane in a mammal, comprising administering to the mammal a composition comprising at least one active agent and at least one enhancer, wherein said enhancer is an acid selected from a group consisting of N-alkylated amino acid, thioctic acid, sebacic acid, shikimic acid, and salts thereof, wherein the enhancer is present in the composition in an amount effective to increase the biological absorption of the active agent.

16. The method of claim 15, wherein said N-alkylated amino acid is a N-methylated amino acid.

17. The method of claim 16, wherein the aminoacid is selected from a group consisting of isoleucine, leucine, lysine, methionine, phenylalanine, threonine, tryptophan, valine, arginine, histadine, alanine, asparagine, aspartate, cysteine, glutamate, glutamine, glycine, proline, serine, and tyrosine.

18. The method of claim 17, wherein the amino acid is glycine.

19. The method of claim 18, wherein said enhancer is selected from a group consisting of N,N-dimethylglycine, trimethylglycine and salts and combinations thereof.

20. The method of claim 19, wherein said enhancer is N,N-dimethylglycine.

21. The method of claim 15, wherein said enhancer is selected from a group consisting of thioctic acid, sebacic acid, shikimic acid, and salts thereof.

22. The method of claim 15, wherein the concentration of the enhancer is from about 0.01% to about 99% by weight.

23. The method of claim 22, wherein the concentration of the enhancer is from about 0.01% to about 50% by weight.

24. The method of claim 23, wherein the concentration of the enhancer is from about 0.1% to about 30% by weight.

25. The method of claim 15, wherein the mucosal membrane is the gastrointestinal tract and the composition is administered orally, buccally or sublingually.

26. A process for preparing the composition of claim 1, comprising bringing into association at least one pharmaceutically active agent with one or more enhancer, optionally adding a carrier, and forming a liquid, suspension, emulsion, aerosol, powder, pill, tablet, capsule, gel caps, troche, cachet or pellet therewith.
